(19) United States
(12) Patent Application Publication
(10) Pub. No.: US 2022/0176121 A1
(43) Pub. Date: Jun. 9, 2022

(54) ADDRESSABLE ELECTRODE ARRAY SYSTEMS, DEVICES AND METHODS

(71) Applicant: MINDMAZE HOLDING SA, Lausanne (CH)

(72) Inventors: Hadrien MICHAUD, Lausanne (CH); Alice TONAZZINI, Lausanne (CH)

(21) Appl. No.: 17/522,924

(22) Filed: Nov. 10, 2021

Related U.S. Application Data
(60) Provisional application No. 63/111,779, filed on Nov. 10, 2020.

(51) Int. Cl. A61N 1/36; A61N 1/04

(52) U.S. Cl. CPC A61N 1/36014; A61N 1/0476

(57) ABSTRACT

A system for an addressable body-worn electrode array, comprising a plurality of electrodes in the array, adapted to support easy and consistent positioning of electrical therapy through the electrodes by the therapist that is retained during therapy.

### FIELD OF THE DISCLOSURE

The present invention relates to addressing electrodes within a body-worn electrode array and, in particular, in relation to functional electrical stimulation (FES) devices.

### BACKGROUND

Functional electrical stimulation (FES) is a popular technique for delivering electrical pulses to generate muscle contractions. FES is used for a number of different indications related to muscular atrophy and motor disorders.

Pathologies of the neuromuscular system due to disease or trauma to the central nervous system, such as stroke, spinal cord injury, head injury, cerebral palsy, and multiple sclerosis, can impede proper functioning of the upper and lower limbs.

Typical FES systems use self-adhering surface electrodes to deliver the pulses and some devices are garment-based where the electrodes are embedded in clothing or in wearable fabric pieces like sleeves or cuffs. Such systems typically provide the wearable component with a receptacle element into which a plug is connected for delivering electrical pulses to the electrodes within the wearable component. The pulse generator includes components that allow for modulation of the pulses as well as switching to address specific electrodes within the wearable component which includes multiple electrodes. Some systems include no switching mechanism and simply provide for statically addressed electrodes, each electrode being wired to a single output channel of the pulse generator.

Although growing clinical evidence shows that FES, and in particular home-based, patient-directed FES, is effective for motor rehabilitation of stroke or brain injury patients, much room remains for the wide adoption of FES as a patient-directed therapy. Prior art FES devices require knowledge of where to place the electrodes and bimanual dexterity for their operation, which are limited in most neurological patients. Moreover, the complexity of use of existing systems deters physical therapists and physicians from using and prescribing FES for home-use.

Stroke or traumatic brain injury often result in arm and hand impairment on one side of the body, on which the patient-directed FES is applied. In the context of a wearable electrode system for patient-directed motor rehabilitation, it is crucial for a patient to be able to don the system with the contralateral, unaffected hand. In addition, it is important for the therapist to position the electrodes based on the morphology and physiologic condition of each patients, to stimulate the correct muscles or nerves involved in therapy. Prior art wearable FES devices failed to deliver high usability for both donning with one hand by the patient and precise positioning of electrodes by the therapist. In the context of home use, the position of the electrodes with respect to anatomical landmarks should be retained for the duration of the therapy, typically several weeks.

For example, the Innovo® from Atlantic Therapeutics™ provides a lower-trunk garment with an array of electrodes, each electrode having a lead wire to a connector built into the waist of the garment. The pulse generator is then plugged into the connector. Upon activation, the pulse generator addresses pulses to individual electrodes. The Innovo® is a single-purpose device for treating incontinence. The static addressing of electrodes at the pulse generator is highly efficient for that purpose. The Innovo® is not useful, however, for other treatments. Moreover, the electrodes activate large muscles and, consequently, occupy large segments of the garment. Importantly, placement of the electrodes and movement of the electrodes during wear relative to the targeted muscles is less of a concern due to the size of the electrodes and the targeted muscles.

The Fesia Grasp® from Fesia Technology™ described in European patent publication 3650077A1 provides a stimulation device for the forearm with an array of electrodes, each electrode having a lead wire to a receptacle built into the garment. The pulse generator is then plugged into the receptacle. Upon activation, the pulse generator addresses pulses to individual electrodes and then delivers the addressed pulses to the electrodes in sequence. The Fesia Grasp® is designed to provide flexion and extension of the wrist and fingers. The therapist can select which electrode to activate using a tablet computer program. However, the therapist setting up the device does not have direct visual cues of the activated stimulation areas and needs to monitor both the screen of the tablet computer and the movements of the patient to adjust the position of stimulation areas through trial and error. This can be especially challenging for therapists that are not familiar with technology and may also require a significant amount of time to setup the device even for therapists that are familiar with technology.

The FES garment described in Moineau et al., Garments for functional electrical stimulation: Design and proofs of concept; J. of Rehab. and Assistive Tech. Eng., 6, 1-15 (2019) (available at https://doi.org/10.1177/2055668319854340) includes conductive textile electrodes for stimulating the forearm, upper arm, and shoulder. However, the shape and relative position of each stimulation area is fixed by design and cannot be adjusted once the garment has been donned. This and other similar prior art devices present would require different sizes, versions, etc. for different patients. Accordingly, the manufacturing process is more costly due to the numerous various versions required for virtually custom fitting for a patient of the device, including different combinations of wearable's size, electrodes placement within the wearable, electrodes' sizes each depending on the patient size and condition. Moreover, the selected electrode lead wires make it impractical for use outside of a laboratory proof-of-concept.

U.S. Pat. No. 4,239,046 to Ong describes an electrode arrangement that uses hook-and-loop fasteners to attach lead wires to a conductive substrate. However, the arrangement is limited to a single electrode and lead wire. The arrangement must be placed at the area of stimulation and, if misplaced, the entire electrode must be displaced to stimulate another area on the skin of a patient.

### SUMMARY OF SOME OF THE EMBODIMENTS

The background art does not teach or suggest an addressable body-worn electrode array that supports easy and consistent positioning of the array portion selected (i.e., electrode array active area) by the therapist. The background art also does not teach or suggest an addressable body-worn electrode array in which the electrode array active area retains its positioning during therapy. The background art also does not teach or suggest a device, such as a garment for example, comprising the addressable body-worn electrode array which is easy for a patient to don, preferably by using a single hand.

The present invention solves several issues of prior art devices and provides: i) configuration of the active electrodes position by a therapist, with the configuration being retained for the duration of the therapy; ii) ease of electrode configuration by the therapist; iii) high usability when a patient dons the system with a single hand.

The present invention allows for the addressing of electrodes by way of a movable conductive addressing pad. A single lead wire runs from the pulse generator to the addressing pad which conducts the stimulation pulse to the electrodes to which it is applied within an array of electrodes.

The addressing pad is shaped according to the needs of the user. Interchangeable addressing pads of differing shapes can be used to address a variety of electrode patterns within the array and a variety of electrodes. The addressing pad can include a fastening element so that its placement over the electrode array is secured. In some cases, the fastening element can be integral to the addressing pad. For example, the addressing pad can include one or more conductive hook-and-loop fastening elements.

Unless otherwise defined, all technical and scientific terms used herein have the same meaning as commonly understood by one of ordinary skill in the art to which this disclosure belongs. The materials, methods, and examples provided herein are illustrative only and not intended to be limiting.

Various embodiments of the methods, systems and apparatuses of the present disclosure can be implemented by hardware and/or by software or a combination thereof. For example, as hardware, selected steps of methodology according to some embodiments can be implemented as a chip and/or a circuit. As software, selected steps of the methodology (e.g., according to some embodiments of the disclosure) can be implemented as a plurality of software instructions being executed by a computer (e.g., using any suitable operating system). Accordingly, in some embodiments, selected steps of methods, systems and/or apparatuses of the present disclosure can be performed by a processor (e.g., executing an application and/or a plurality of instructions).

Although embodiments of the present disclosure are described with regard to a “computer,” and/or with respect to a “computer network,” it should be noted that optionally any device featuring a processor and the ability to execute one or more instructions is within the scope of the disclosure, such as may be referred to herein as simply a computer or a computational device and which includes (but not limited to) any type of personal computer (PC), a server, a cellular telephone, an IP telephone, a smartphone or other type of mobile computational device, a PDA (personal digital assistant), a thin client, a smartwatch, head mounted display or other wearable that is able to communicate wired or wirelessly with a local or remote device. To this end, any two or more of such devices in communication with each other may comprise a “computer network.”

### BRIEF DESCRIPTION OF THE DRAWINGS

Embodiments of the disclosure are herein described, by way of example only, with reference to the accompanying drawings. With specific reference now to the drawings in detail, it is stressed that particulars shown are by way of example and for purposes of illustrative discussion of the various embodiments of the present disclosure only and are presented in order to provide what is believed to be a useful and readily understood description of the principles and conceptual aspects of the various embodiments of inventions disclosed therein.

FIG. 1 illustrates a schematic for an addressable electrode array as viewed from the bottom in accordance with embodiments.

FIG. 2 illustrates a schematic for an addressable electrode array as viewed from the top in accordance with embodiments.

FIG. 3 illustrates a side-view schematic of an addressable electrode array in accordance with embodiments.

FIG. 4A and FIG. 4B illustrate side-view schematics of a portion of an addressable electrode array in accordance with embodiments.

FIG. 5 illustrates an arrangement of sleeves in accordance with embodiments.

FIGS. 6A-6E illustrate side-view schematics of an addressable electrode array during manufacturing steps in accordance with embodiments.

FIG. 7 illustrates a schematic for a 6×5 addressable electrode array as viewed from the bottom in accordance with embodiments.

FIG. 8 illustrates a schematic for a pseudo-circular addressable electrode array as viewed from the bottom in accordance with embodiments.

FIG. 9 illustrates a schematic for an addressable electrode array having varying sizes of electrodes as viewed from the bottom in accordance with embodiments.

FIG. 10 illustrates a schematic for an addressable electrode array and replaceable addressing pads as viewed from the top in accordance with embodiments.

FIG. 11, in particular with regard to FIGS. 11A-11E, illustrates various addressing pads patterns in accordance with embodiments.

FIG. 12 illustrates an exemplary, non-limiting method for use of an exemplary device as described herein for therapy.

### DETAILED DESCRIPTION OF SOME OF THE EMBODIMENTS

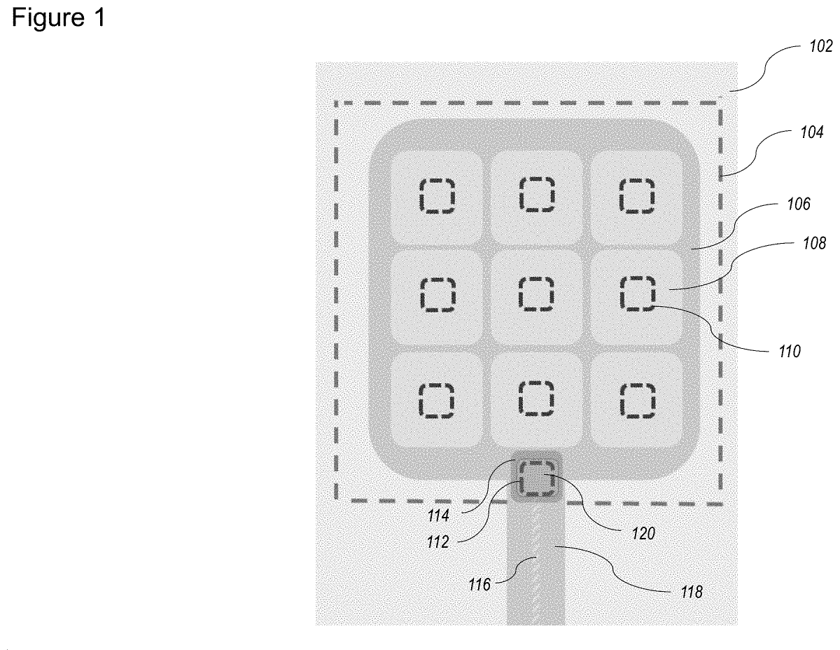

FIG. 1 illustrates a schematic for an addressable electrode array as viewed from the bottom, or the side to be in contact with the skin, in accordance with embodiments. A substrate 102 is included which in preferred embodiments is made of a flexible or stretchable fabric. An insulation 106 is layered above the substrate 102 and in which electrodes 108 are embedded. In some preferred embodiments, the insulation 106 is composed primarily or entirely of polyurethane. The insulation 106 can be composed of or include other materials including silicone, butyl rubber, neoprene, nitrile rubber and the like. The thickness of the insulation 106 is preferably between 1 μm and 1 mm. In preferred embodiments, the insulation 106 has substantially the same conformability and stretchability as the substrate 102. The electrode 108 and insulation 106 stack should be less stiff than the substrate to keep at least the same range of flexibility as the substrate 102. The distance between electrodes is preferably between 0.1 mm and 10 mm.

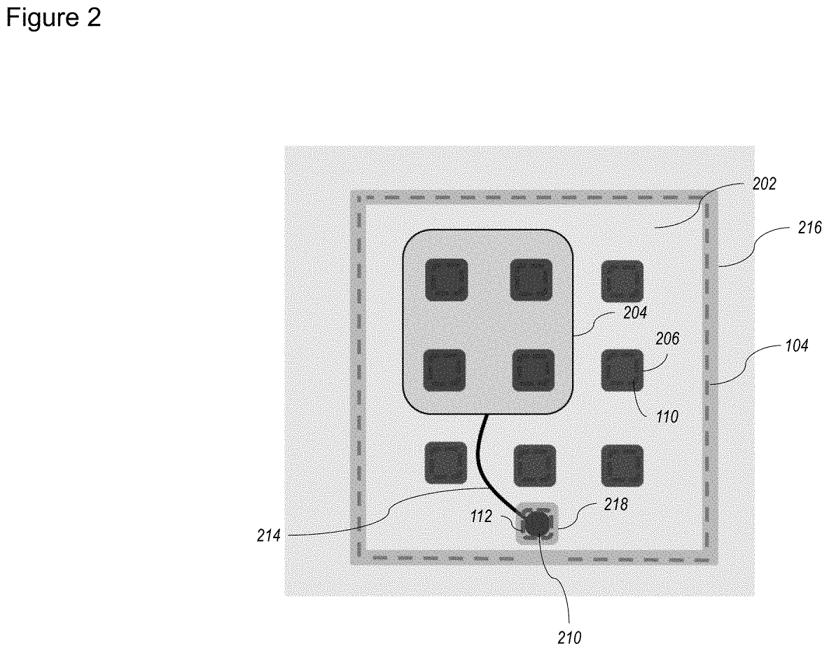

Surrounding the insulation 106 is a nonconductive threading 104 that is sewn through to the opposing side. The nonconductive threading 104, as will be discussed further in connection with FIG. 2, attaches a hook-and-loop face, a zipper, a series of snaps or buttons or any other type of fastening mechanism known in the art to the opposing side. A conductive threading 110 is sewn through each electrode 108 to the opposing side. The conductive threading 110 can be of silver-coated polyamide or polyester, copper-coated polyamide or polyester, gold-coated polyamide or polyester, stainless steel, or other appropriate fabric or threading. Conductive threading 110 preferably has a linear resistance between 1 and 1000 Ω/m. The array shown includes nine electrodes 108. An electrode 108 is preferably an elastomer loaded with conductive particles. The elastomer can be polyurethane, silicone, butyl rubber, neoprene, nitrile rubber or similar polymer. The conductive particles can include carbon, silver, copper, gold, platinum, platinum-iridium alloy, indium tin oxide, carbon nanotubes, graphene, and the like.

An electrode 108 can also be made of conductive textile including silver-coated fabric, copper-coated fabric, stainless-steel fabric, of hydrogels, or of multilayer materials made of hydrogel layers and conductive composite or conductive textile layers.

Electrodes 108 can be of various shapes such as circular, ovular, polygonal with three or more sides, e.g., triangles, rectangles, hexagon, etc., and can be arranged in various patterns to form an array (e.g., matrix, honeycomb, etc.). Preferably, where the array includes multiple electrodes, the pattern arrangement is dense. This can allow for more precise activation of electrodes within the array. Each array or group of electrodes can include two or more electrodes. The area of an individual electrode is preferably between at least 5 mm² and 100 mm². The area of an electrode array active area thus preferably has an area lower bound of at least about 2×5 mm² plus spacing between electrodes and, similarly, preferably has an area upper bound 2×100 mm² plus spacing where electrodes have the same size, or the size of at least two electrodes at the upper bound of the size of an electrode. The area of an addressing pad likewise preferably has an area lower bound of at least about 2×5 mm². In some embodiments the array of electrodes in the device can be of different sizes and dimensions and, thus, the bounds of the electrode array active area can be smaller. The main determining factor for the area of the electrode is the size of the muscle group targeted. In cases where a larger muscle area is targeted there may be larger electrodes toward the center of the electrode array and smaller electrodes away from the center for example. Smaller electrodes and addressing pads/electrode array active areas are preferably used in devices targeting smaller areas, e.g., the hand, while the larger electrodes and addressing pads/electrode array active areas are preferably used in devices targeting larger muscle areas, e.g., upper arm, upper leg.

The above dimension bounds apply regardless of shape of the electrode. That is, whether square or non-square, an electrode preferably has at least a 5 mm central line (i.e., line through the center point) at its narrowest. For example, circular electrodes preferably have a diameter of at least 5 mm, ovular electrodes preferably have a minor axis of 5 mm, and hexagons preferably have a central line of 5 mm at its narrowest. The above dimensions are preferable in most embodiments but dimensions outside the discussed ranges can be used depending on the circumstances in accordance with embodiments of the invention, including electrodes having dimensions smaller than 5 mm×5 mm. In prior art devices, smaller electrodes are impractical and, in some cases, dangerous because current would be directed to such a small area. For example, a user may experience burning with small electrodes. In some embodiments of the present invention, however, addressing pads can be larger than individual electrodes so that larger areas are targeted and, thus, those limitations in the prior art are obviated. It should also be understood that in some embodiments the area covered by the electrode array active area is only substantially equal to the area covered by the addressing pad and in some embodiments it can differ significantly. In some cases, the addressing pad can be larger, for example to allow for a fastener on the addressing pad that extends beyond the electrode array active area, or smaller. Preferably, the addressing pad shape and area are substantially similar to the electrodes array active array to assist the user in addressing the proper electrodes.

The total size of the electrode array in the device and electrode array active area in each instance will depend on the use case. That is, the size of the user and the treatment to be performed, among other factors, will determine the most appropriate electrode active area size, dimensions and shape. For example, an electrode array to be used on large muscles of the upper arm or upper leg typically will be larger than arrays and electrode array active areas intended for use on the lower arm or lower leg, respectively. As noted above, a wearable electrode array device for a small muscle group (e.g., hand muscles) can use a smaller electrode and smaller electrode array active area such as 2×2 electrode array active area having 5 mm×5 mm electrodes. Regardless of the size of the electrode array active area or the number of electrodes within it, there are preferably additional electrodes in the device. For example, in a wearable electrode array device for the hand, the number of electrodes in the device can range between 2×3 and the number of electrodes required to encompass the surface area of the device plus spacing between the electrodes. For larger muscle areas, such as the upper arm or upper leg, preferred embodiments include a larger electrode array active area, or addressing pad, for example, 3 cm×4 cm or 5 cm×5 cm active area with larger electrodes, for example, 2.5 cm×2.5 cm. A 4-electrode array of this size would be preferable for large muscle groups for most users in most cases. In some embodiments, the total area of an electrode array can be up to the total surface area of the targeted muscles. For example, the electrode array of a device targeting the upper arm typically would have a surface area around 450 cm² with an electrode array active area surface area around 225 cm² (about 15 cm×15 cm for a square-shaped electrode array active area). However, again, the size of the respective surface areas depends on the size of user and the use case and in the case there functional stimulation would be useful for much larger-than-average individuals. Similarly, the pattern of the electrode array and the electrode array active area/addressing pad preferably are suited to the intended use of that electrode array.

Returning to FIG. 1, a skin-side lead junction 120 is insulated by way of insulation 106 from the electrodes in the substrate. Unlike in much of the prior art, each electrode is insulated and unconnected to any lead wiring through the substrate. Lead junction 120 is enclosed by an insulation patch 114 to protect it from exposure because the lead junction is conductive. From lead junction 120 is an insulated lead wire 116 to a pulse generator which is not shown in the figure. The lead wire 116 is enclosed in wire insulation 118. Wire insulation 118 can be of the same materials as the insulation 106.

The pulse generator can be any known pulse generator. In preferred embodiments, however, the pulse generator is a transcutaneous electrical stimulation device, such as the Intento PRO pulse generator or RehaStim 2® (Hasomed™), Rehab X2® (Cefar™), or AvivaStim® (Saebo™)

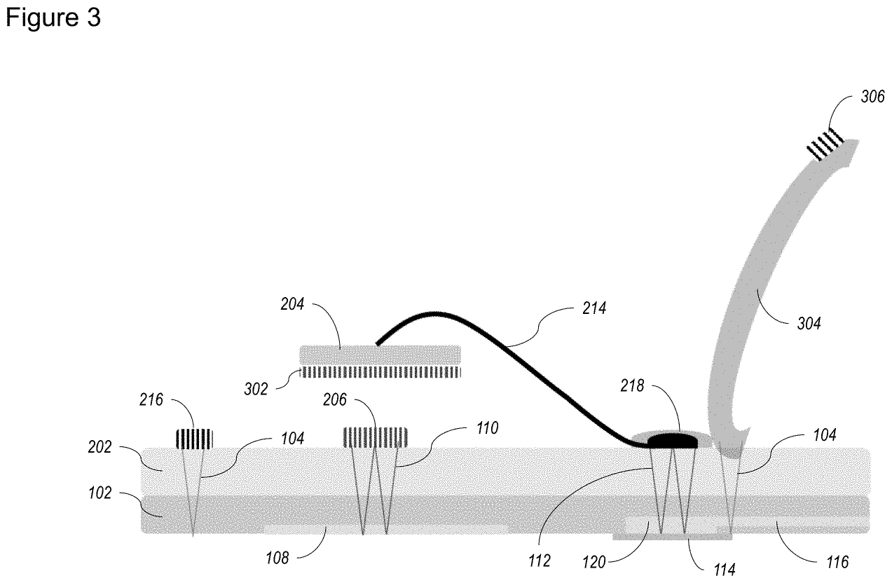

Turning now to FIG. 2, an illustrative schematic of the opposing side of the addressable electrode array in accordance with embodiments is shown. Surrounding the electrode array is a fastening perimeter 216. Fastening perimeter 216 allows a cover flap (not shown) to be placed over the electrode array to protect and insulate any of the exposed conductive elements while in use. Fastening perimeter 216 can be hook-and-loop, zipper, a series of snaps or buttons or any other type of fastening mechanism known in the art. In the embodiment illustrated, a hook-and-loop fastener is used and is sewn around the perimeter of the electrode array with nonconductive threading 104. In the embodiments shown in FIG. 3, nonconductive threading 104 penetrates the entire thickness of the conductive and insulating layers. Conductive selection patches 206 are conductively connected to the electrodes 108 on the opposing side via conductive threading 110. In preferred embodiments, addressing pad 204 use a reversible fastening technology to attach to the conductive selection patches 206 so that detachment is made simple and the conductive addressing pad 204 can be repeatedly attached and detached. The preferred fastening technology is hook-and-loop but could also include magnet, reusable adhesives, zipper, snap, through-fastener (e.g., button, lacing, or other fastening device that fastens through the material). The fastening component provides the conductivity in preferred embodiments. In some cases, however, the conductive selection patch 206 can include a non-conductive fastener and a separate conductive component or material attached to or integral with the conductive selection patch 206 and that maintains contact with the electrode array during use. A combination of conductive and non-conductive fastening components can be used as well. An example of a preferred conductive selection patch 206 is Conductive Hooks and Loops from Kitronik Ltd.

Conductive selection patches 206 preferably have a linear resistance of 1 Ω/cm for a 2.5 cm wide strip and are preferably made of silver-coated plastic. A linear resistance of less than 5 Ω/cm for a 2.5 cm wide strip would be acceptable. A lead junction 210 is conductively connected to skin-side lead junction 120 via conductive threading 112. A lead wire 214 is run from the lead junction 210 to an addressing pad 204 which is applied to one or multiple conductive selection patches 206. The conductive fastener 302 of the addressing pad 204 shown in FIG. 3 that is applied to conductive selection patches 206 is made from an electrically conductive material which is connected to lead wire 214. In some embodiments, the entirety of the addressing pad 204 can be a single layer of conductive material. As noted above and discussed further below, an insulating patch is applied to the top of the array of conductive selection patches 206 to protect any exposed conductive elements while in use. In some embodiments, the addressing pad 204 can include an insulating layer for further protection. In the embodiment shown, the conductive selection patches 206 and addressing pad 204 use a conductive hook-and-loop fastener for applying and securing the addressing pad 204. Some embodiments may use other conductive fasteners such as snaps, magnets, and the like.

In operation, the stimulation pulse from the pulse generator is delivered to the addressed electrodes 108, on the opposing side from the addressing pad 204 through conductive path described above: insulated lead wire 116, skin-side lead junction 120, conductive threading 112, lead junction 210, lead wire 214, addressing pad 204, addressed conductive selection patch 206, conductive thread 110, addressed and electrode 108. Prior art devices include switches at the pulse generator to activate lead wires permanently connected to specific electrodes. For garment-based electrodes however the problem of electrode placement to generate contraction of a specific muscle or muscle group remains. Thus, prior art devices are left with either providing large electrodes to account for inaccurate placement and movement but that cannot target a specific muscle group or providing complex switching mechanisms at the pulse generator. In contrast with the prior art, electrodes are addressed here by way of placing the addressing pad 204 on the conductive selection patches in correspondence of the electrodes to be activated. This allows for users to easily switch the set of active electrodes within the array.

Prior art devices and systems suffer from the following complications which are alleviated by embodiments described. In prior art devices, visualizing the stimulated area requires a screen or other representation. On the contrary, with embodiments described herein, the addressing pad is placed in visual and physical correspondence with the stimulated area. Many prior art devices can stimulate some areas inadvertently (e.g., because of incorrect manipulation, software bugs, and the like) without a therapist or patient realizing. On the contrary, with embodiments described herein, the addressing pads are physically displaced by a user as they would do with standard self-adhering electrodes, limiting the risk of incorrect manipulation. Furthermore, many prior art devices require the user (e.g., therapist) to learn a new procedure to select the correct active electrode site. Embodiments described herein solve this problem by allowing the user to place the addressing pads in a trial-and-error procedure that is very similar to the procedure they have been trained for with standard self-adhering electrodes.

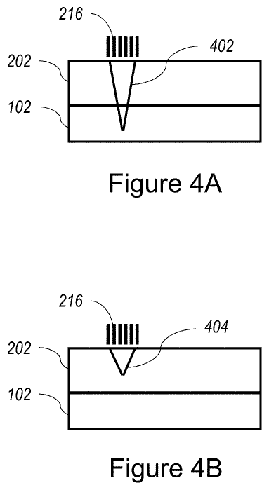

FIG. 3 illustrates a side-view schematic of an addressable electrode array in accordance with embodiments. Conductive threading 110 and 112 and nonconductive threading 104 are shown passing through from the base side to the opposing side of the device. As can be seen in FIGS. 4A and 4B, nonconductive threading need not pass through the entire depth of the device. For example, as illustrated in FIG. 4A, nonconductive threading 402 can pass through at least partially each layer. In another example, as illustrated in FIG. 4B, nonconductive threading 404 can partially pass through a single layer. Nonconductive threading should pass deep enough into the material so that fastener 216 can remain attached to the opposing side through repeated use. It should be understood that nonconductive threading may not be required. For example, an adhesive, heat transfer, or ultrasonic welding may be used. Additionally, in some embodiments, a type of fastener can be used that does not require threading.

Returning to FIG. 3, conductive fastener 302 is shown. Additionally, protective insulating cover 304 with nonconductive fastener 306 is shown. In the embodiment illustrated in FIG. 3, nonconductive fastener components 216 and 306 are hook-and-loop. In some embodiments, as explained above in relation to conductive fasteners, other types of fastening components can be used. Layers 102 and 202 are shown. It should be understood that other layers may be included.

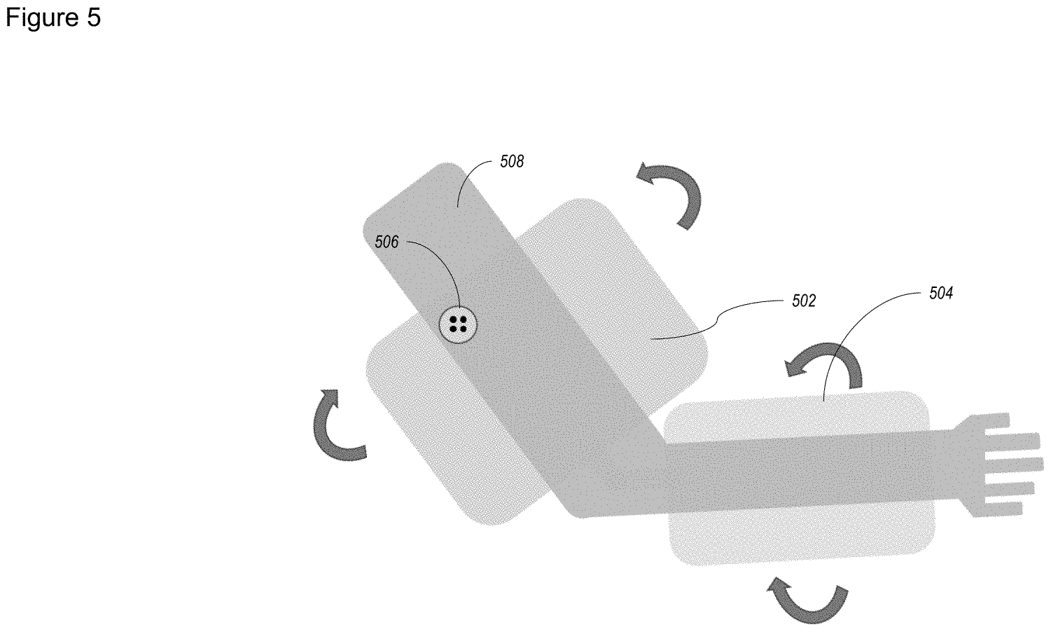

FIG. 5 illustrates an arrangement of sleeves in accordance with embodiments. Shown are sleeves 502 and 504. Embodiments can include one or more sleeves. In the embodiment shown, sleeves 502 and 504 attach to the upper and lower arm, respectively, by wrapping around the arm 508 at least partially. Embodiments can include fasteners on the sleeves to attach one end to the other to maintain compression on the limb and thereby maintain position on the limb. Fastener components to connect sleeve ends can include fasteners as discussed elsewhere herein. Embodiments can include electrode arrays in a pad format in which the pad itself does not wrap around at least partially to maintain position but adheres to a limb or body part with an adhesive, strap or other attached fastener component. Embodiments can include alignment marks to align the sleeves with anatomical landmarks to maintain precise positioning of the electrode arrays with respect to the user's muscle across multiple donning and doffing. At the top end of the electrode array device is a connector housing 506 that allows connection to the pulse generator. The connector 506 may be placed anywhere on the sleeves and several connectors may be distributed on the sleeves.

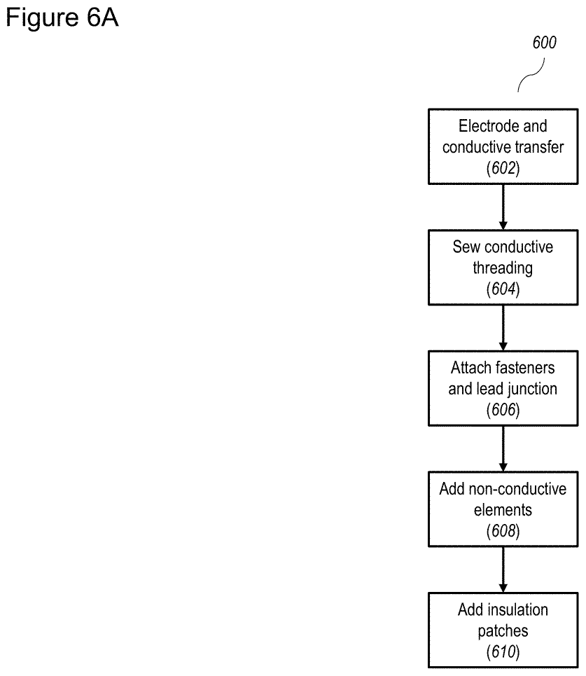

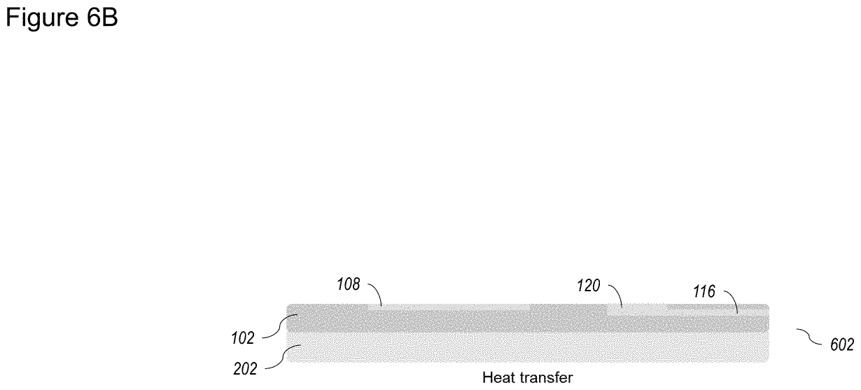

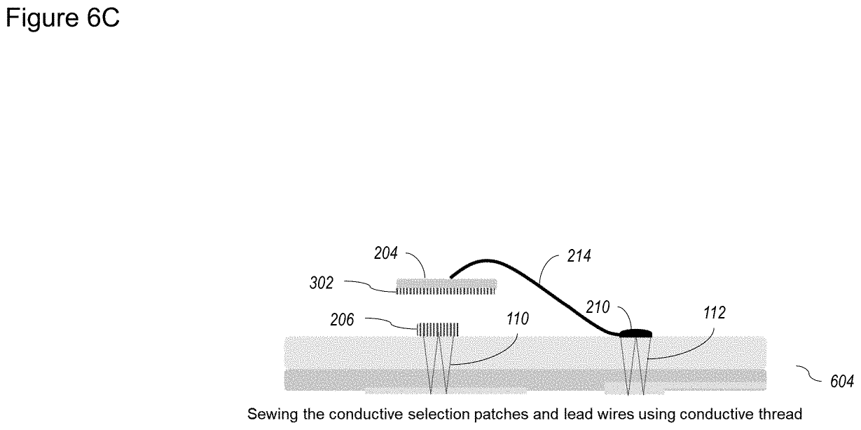

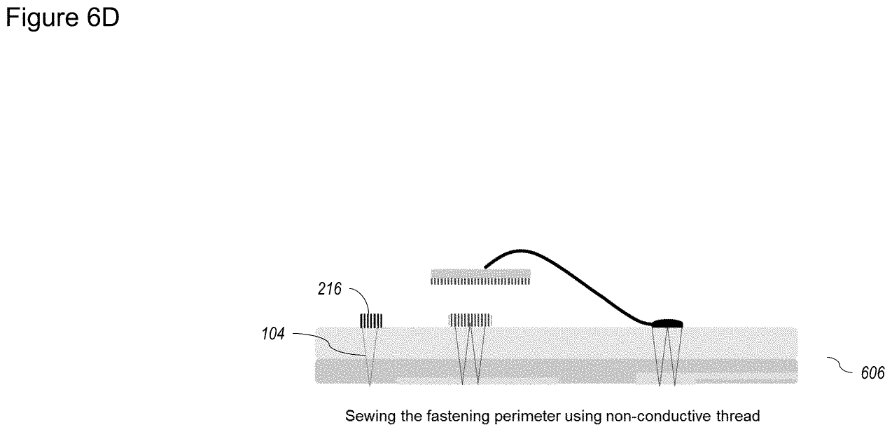

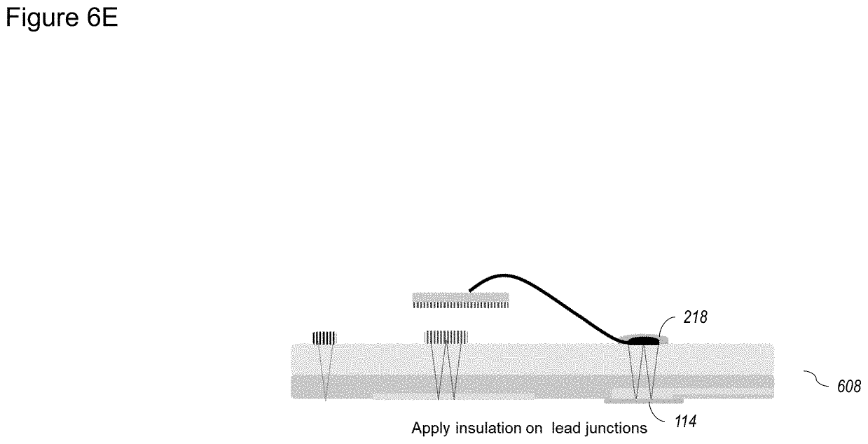

FIGS. 6A-6E illustrate side-view schematics of an addressable electrode array during manufacturing steps in accordance with embodiments. FIG. 6A shows the overall process as a method, while FIGS. 6B-6E illustrates each stage schematically in more detail. At 602 and FIG. 6B, the electrodes 108 and conductive traces 116 are transferred onto layer 202. Preferably, this is done using heat transfer. At 604 and FIG. 6C (partially), conductive threading 110 and 112 are sewn into the electrode array layers. At 606 and FIG. 6C (partially) the conductive fasteners 206 and lead junction 210 are attached to the opposing side material, thereby providing a conductive trace to the electrode 108 and lead junction 120. At 608 and FIG. 6D, nonconductive elements such as nonconductive threading 104 and nonconductive fasteners 216 are added. At 610 and FIG. 6E, insulation patches 114 and 218 are added to cover and protect conductive material, such as lead junctions 120 and 210.

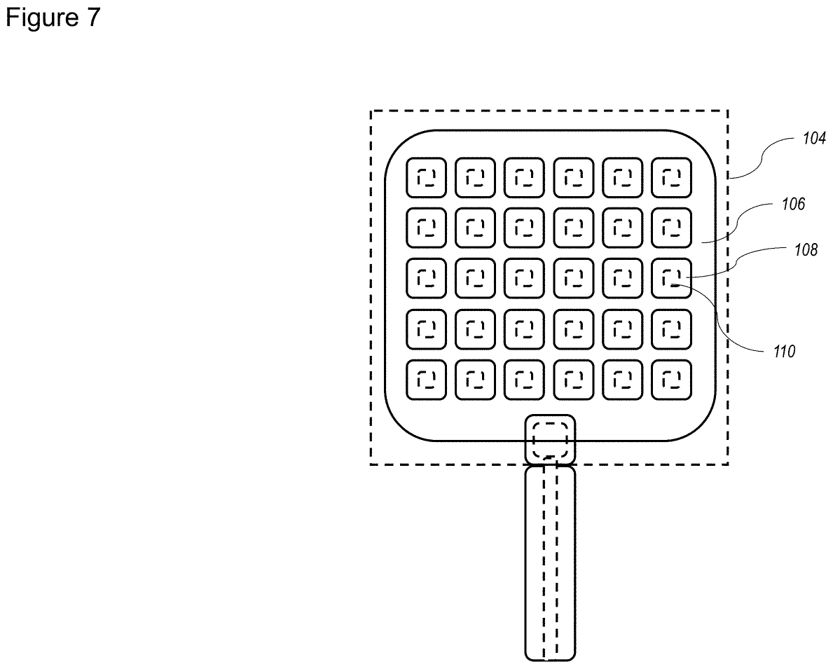

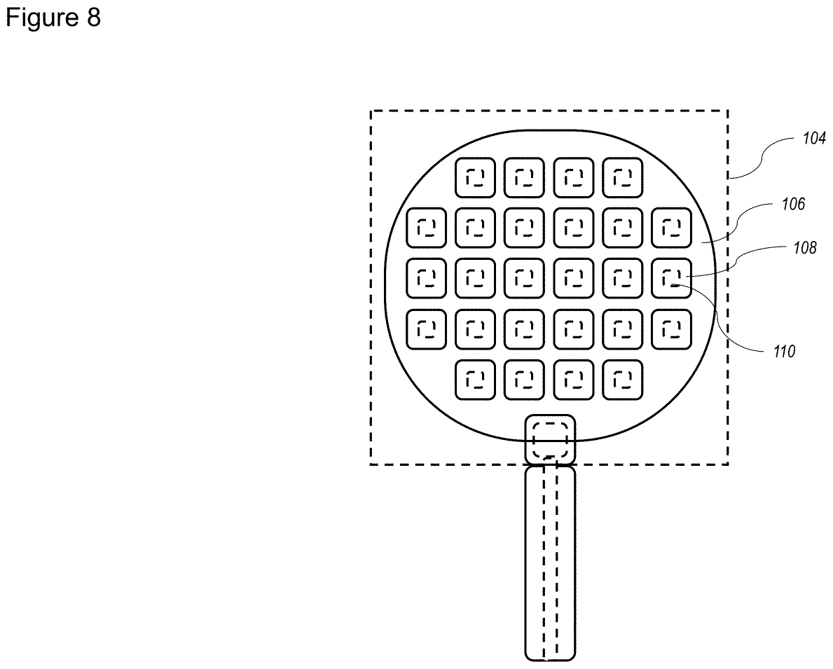

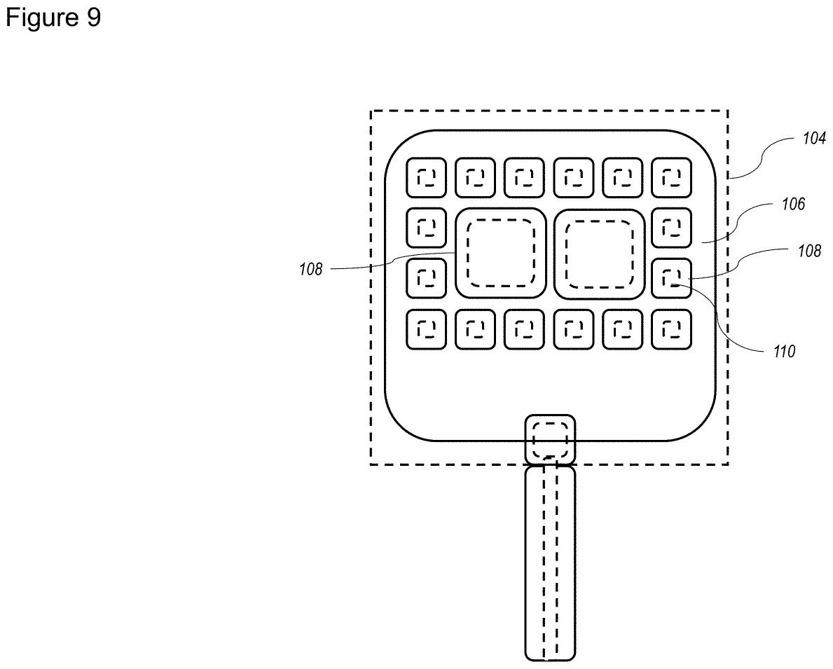

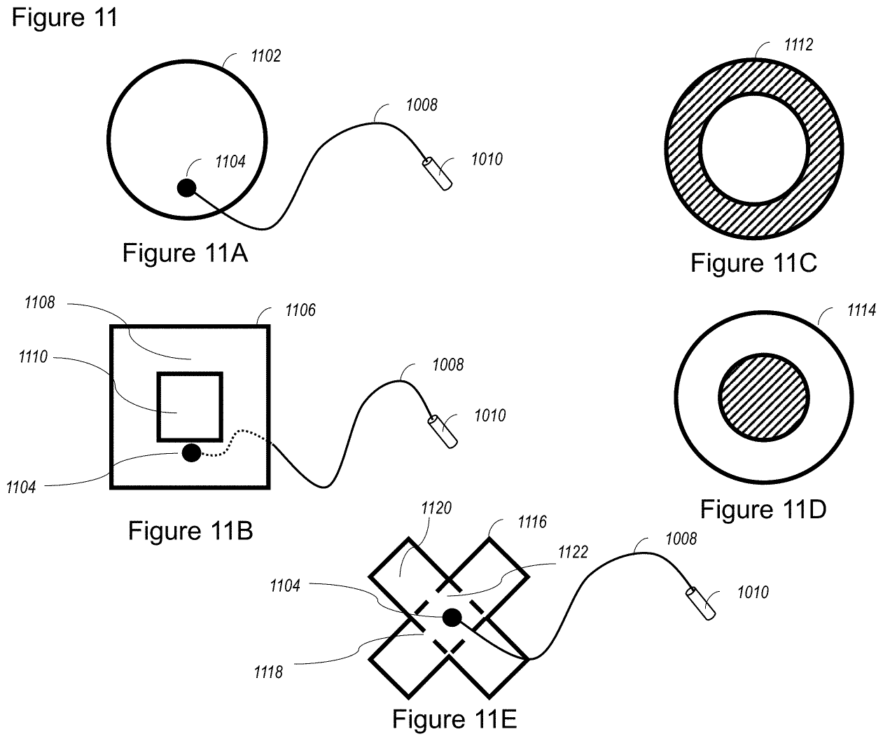

FIG. 7 illustrates a schematic of a 6×5 electrode array as viewed from the bottom, or the side to be in contact with the skin, in accordance with embodiments. One or more of the electrodes 108 can be activated using addressing pads discussed herein and, in particular, below in connection with FIGS. 11A-11E. Arrays of varying dimensions and resolution, and with varying electrode sizes can be used in accordance with embodiments. FIG. 8 illustrates a schematic of an array in a pseudo-circular format in accordance with embodiments. FIG. 9 illustrates a schematic of an electrode array in accordance with embodiments having varying sizes of electrodes. As discussed herein, variable sized addressing pads or addressing pads with different geometries can be used to select the active electrodes.

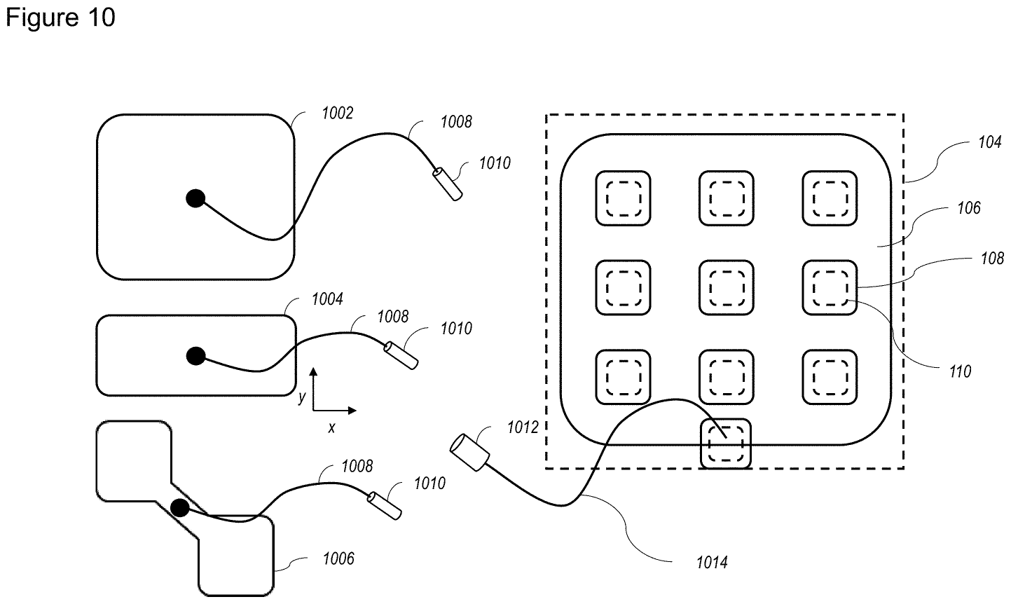

FIG. 10 illustrates a schematic of an electrode array and replaceable addressing pads for use thereon and having different geometries to actively select various electrodes for stimulation, as seen from the top, or the side opposing to the skin, in accordance with embodiments. In the schematic shown, addressing pad 1002 can be used to select four electrodes in a 2×2 subarray of the array. Addressing pad 1004 can be used to select two electrodes in a 1×2 subarray, either along the x axis or the y axis. And addressing pad 1006 can be used to select two catercorner electrodes. Each addressing pad includes a lead wire 1008 with a detachable connector 1010 to connect to connector 1012 and lead wire 1014 to provide a conductive path from the pulse generator to the electrodes 108.

In some embodiments, multiple addressing pads can be used to address electrodes in an array in lieu of relying on specific shapes of addressing pads. For example, a first and a second addressing pads can be combined to address electrodes in the way one of addressing pads 1002, 1004, 1006 addresses electrodes. In some instances, additional addressing pads beyond a second can be combined. In such cases, each of the addressing pads preferably include lead wires to provide a conductive path. In some instances, the lead wires can connect to the connector 1012 or the lead wires can be connected to similar connectors on other addressing pads to create a conductive path.

FIGS. 11A-11E illustrate various addressing pads patterns in accordance with embodiments. The geometries of addressing pads can correspond to the electrode array size, dimension, and geometry. The addressing pad 1102 of FIG. 11A is circular. As noted, any geometry is possible based on the electrode array. Addressing pad 1102 includes an offset lead wire junction 1104 which can allow for easier removal of the addressing pad and lead to less wear in the area of the lead wire junction.

The addressing pad 1106 of FIG. 11B includes a concentric square design having an outer square 1108 and inner square 1110. The inner square 1110 and the area of the outer square 1108 outside of the area of the inner square can be covered by an insulating layer. On use, one or both insulating layers can be removed to expose only those areas to be used to select electrodes. In preferred embodiments, the insulating layer is a thin shielded layer attached to the conductive fastener 302 of the addressing pad 204 using either the same fastener component used for the conductive selection patch or a different fastener appropriate for the material of the face of the addressing pad. For example, if the addressing pad uses a magnetic layer conductivity, an adhesive or magnetic material can be used to attach the insulating layer. Lead wire junction 1104 is shown as it would attach to the opposing side in a way that it connects to both sections of the addressing pad. In some embodiments, each section can have its own lead wire junction. Addressing pads 1112 and 1114 of FIGS. 11C and 11D, respectively, illustrate schematics of addressing pads similar to that of FIG. 11B although with a circular geometry. The shaded areas illustrate insulated areas of the patches so that only the inner circle (FIG. 11C) or only the annulus (FIG. 11D) are exposed and thus used to select electrodes. The lead wire junctions and lead wires are not shown but can be configured as described in connection with FIG. 11B.

The addressing pad 1116 of FIG. 11E illustrates a foldable addressing pad. Fold lines 1118 indicate where the conductive material can be folded away from the electrodes or unfolded to deselect or select electrodes. As shown, lead wire junction 1104 is in the center of the addressing pad 1116. In preferred embodiments, a hook-and-loop or other fastener can be used to fold a foldable section 1120 onto the base section 1122 to maintain the addressing pad in a given shape and prevent connection with adjacent selection patches. To accommodate attachment and the lead wire 1008, a notch can be made in the edge of the foldable section 1120. In some embodiments, the protective cover (not shown) as described above can hold foldable sections 1120 in place to prevent inadvertent selection of an adjacent electrode.

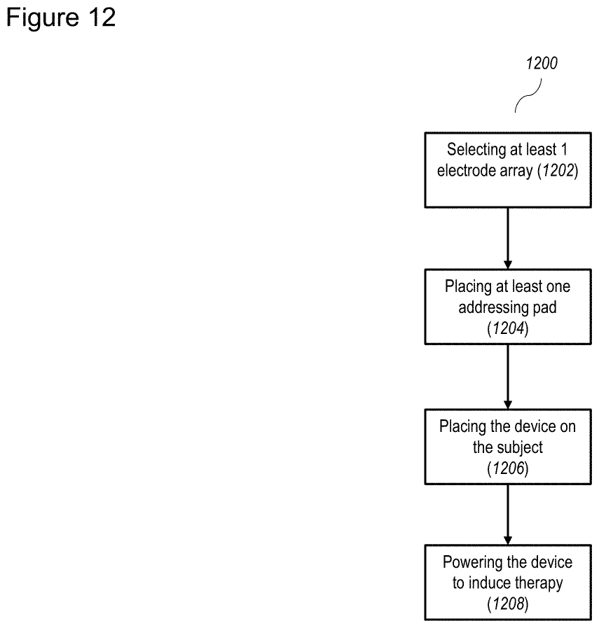

FIG. 12 illustrates an exemplary, non-limiting method for use of an exemplary device as described herein for therapy. As shown in a method 1200, the method begins by selecting at least one electrode array portion for delivering electrical therapy at 1202. A therapist may perform such a selection, for example according to which muscle is to receive electrical therapy. At 1204, at least one addressing pad is placed on the device to deliver the electrical therapy, in which the position of the addressing pad corresponds to the at least one electrode array portion selected (i.e., electrode array active area) in the correct position to deliver therapy. Adding addressing pad to permit electrical therapy to be delivered is a safer option, in that the default is that therapy is not delivered at a particular position without such an addressing pad.

At 1206, the device is placed on the body of the subject. For example, the device may be incorporated to a garment that is worn by the subject, in such a manner that the electrode array portion that is able to deliver electrical therapy is in the correct position. At 1208, power is supplied to the device to begin therapy; however, electrical therapy is only delivered to the position or positions of electrodes at which an addressing pad was placed. Without wishing to be limited by a single hypothesis, the addressing pads allow for addressing certain electrodes at the wearable instead of at the generator (power supply). They are easier for patients to manage at home than addressing using the pulse generator interface (e.g., the addressing pads are a visual and manual addressing device and, thus, intuitive whereas using an interface at the generator requires some special knowledge of how the generator and addressing work).

Any and all references to publications or other documents, including but not limited to, patents, patent applications, articles, webpages, books, etc., presented in the present application, are herein incorporated by reference in their entirety.

Example embodiments of the devices, systems and methods have been described herein. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and apparatuses which may further include any and all elements from any other disclosed methods, systems, and apparatuses, including any and all elements corresponding to target particle separation, focusing/concentration. In other words, elements from one or another disclosed embodiment may be interchangeable with elements from other disclosed embodiments. In addition, one or more features/elements of disclosed embodiments may be removed and still result in patentable subject matter (and thus, resulting in yet more embodiments of the subject disclosure). Correspondingly, some embodiments of the present disclosure may be patentably distinct from one and/or another reference by specifically lacking one or more elements/features. In other words, claims to certain embodiments may contain negative limitation to specifically exclude one or more elements/features resulting in embodiments which are patentably distinct from the prior art which include such features/elements.

What is claimed is:

1. A system for an addressable body-worn electrode array, comprising a plurality of electrodes in the array, adapted to support easy electrical therapy through consistent positioning of the electrodes by the therapist that is retained during therapy.

2. The system of claim 1, further comprising an apparel device for being worn on the body, wherein the apparel device comprises the electrode array, the apparel device further comprising a plurality of addressing pads, wherein placement of the addressing pads determines positioning of active electrodes.

3. The system of claim 1, wherein the array further comprises a substrate that is at least one of flexible and stretchable for attaching, embedding or integrally forming the electrodes.

4. The system of claim 3, wherein the electrode array further comprises a movable conductive pad for positioning the active electrodes.
